(19) United States
(12) Patent Application Publication
(10) Pub. No.: US 2010/0204770 A1
(43) Pub. Date: Aug. 12, 2010

(54) Stent Delivery System Permitting in Vivo Stent Repositioning

(75) Inventors: Juan-Pablo Mas, Indianapolis, IN (US); Nareak Douk, Lowell, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/368,419

(22) Filed: Feb. 10, 2009

(51) Int. Cl. A61F 2/06; A61M 25/00

(52) U.S. Cl. 623/1.11; 604/264; 623/1.12

(57) ABSTRACT

A stent delivery system is disclosed that includes a recapture component for at least partially collapsing an improperly deployed stent in situ to permit repositioning and re-deployment of the stent. The recapture component and the stent may be disconnected in situ to allow for removal of the stent delivery system. In one embodiment, the recapture component is a balloon having loops or hooks around a periphery thereof for receiving at least one removable tether that couples the balloon and stent together. In another embodiment, the recapture component is an expandable tubular component connected to the stent via the removable tether.

### FIELD OF THE INVENTION

The present invention is directed to endoluminal prostheses for use in a body lumen. More particularly, the present invention is directed to a stent delivery system including a recapture component for partially collapsing a deployed stent in situ to permit repositioning of the stent.

### BACKGROUND OF THE INVENTION

A wide range of medical treatments are known that utilize “endoluminal prostheses.” As used herein, endoluminal prostheses are intended to mean medical devices that are adapted for temporary or permanent implantation within a body lumen, including both naturally occurring and artificially made lumens. Examples of lumens in which endoluminal prostheses may be implanted include, without limitation: arteries, such as those located within the coronary, mesentery, peripheral, or cerebral vasculature; veins; gastrointestinal tract; biliary tract; urethra; trachea; hepatic shunts; and fallopian tubes.

Various types of endoluminal prostheses are also known, each providing a component for modifying the mechanics of the targeted luminal wall. For example, stent prostheses are known for implantation within body lumens for providing artificial radial support to the wall tissue, which forms the various lumens within the body, and often more specifically within the blood vessels of the body.

To provide radial support to a blood vessel, such as one that has been widened by a percutaneous transluminal coronary angioplasty, commonly referred to as “angioplasty,” “PTA” or “PTCA”, a stent is implanted in conjunction with the procedure. Under this procedure, the stent may be collapsed to an insertion diameter and inserted into a body lumen at a site remote from the diseased vessel. The stent may then be delivered to the desired treatment site within the affected lumen and deployed, by self-expansion or radial expansion, to its desired diameter for treatment.

Recently, flexible stented valve prostheses and various delivery devices that can be delivered transvenously using a catheter-based delivery system have been developed for heart and venous valve replacement. These stented valves include a collapsible prosthetic valve attached to the interior of a tubular frame or stent, which can be either self-expanding or balloon expandable. The stented valves can also include a non-porous tubular portion or “stent graft” that can be attached to the interior or exterior of the stent to provide a generally tubular internal passage for the flow of blood when the valve leaflets are open. The graft can be separate from the valve and it can be made from any suitable biocompatible material including, but not limited to, fabric, a homograft, porcine vessels, bovine vessels, and equine vessels. The stented valve can be reduced in diameter, mounted on a catheter, and advanced through the circulatory system of the patient. Once the stented valve is positioned at the delivery site, the stent frame is expanded to hold the valve firmly in place. The prosthetic valve survives the compression and subsequent expansion in fully working form. One embodiment of a stented valve is disclosed in U.S. Pat. No. 5,957,949 to Leonhardt et al. entitled “Percutaneous Placement Valve Stent”, which is incorporated by reference herein in its entirety.

In all stent applications, particularly in stented valve applications, a fundamental concern is that the prosthesis be deployed in the vessel at the target location as precisely as possible. In an application where a stent is used to deliver therapeutic radiation to a target location, proper positioning of the prosthesis is vital to the efficacy of the treatment. However, accurate positioning of the stent prosthesis may be difficult due to complexities in the anatomy as well as other factors, and an initial deployment of the stent prosthesis may result in a less than optimal positioning or, even worse, an inoperable positioning. Thus there is a need in the art for a stent delivery system that permits in situ repositioning of a deployed stent that is less that optimally or improperly positioned.

### BRIEF SUMMARY OF THE INVENTION

Embodiments herein are directed to a stent delivery system for repositioning a deployed stent in situ. The stent delivery system includes a recapture component movable between a collapsed configuration and an expanded configuration. A deployable stent is connected to the recapture component such that the stent, after deployment may be at least partially collapsed when the recapture component is moved to the collapsed configuration. The stent and recapture component may be disconnected from each other in situ to allow for removal of the stent delivery system.

### BRIEF DESCRIPTION OF DRAWINGS

The foregoing and other features and advantages of the invention will be apparent from the following description of embodiments thereof as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a side view schematic of a stent delivery system according to an embodiment hereof.

FIG. 2 is a cross-sectional view of FIG. 1 taken along line A-A of FIG. 1.

FIG. 3 is a side view of the balloon of the stent delivery system depicted in FIG. 1.

FIG. 3A is a cross-sectional view of FIG. 3 taken along line X-X of FIG. 3, according to one embodiment hereof.

FIG. 3B is a cross-sectional view of FIG. 3, according to an alternate embodiment hereof.

FIG. 4 is a distal portion of a stent delivery system according to an alternate embodiment hereof.

FIG. 5 is a side view schematic of a stent delivery system according to an alternate embodiment hereof.

FIG. 6 is an enlarged view of a recapture device having an expandable tubular component according to another embodiment hereof.

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to limit the invention or the application and uses of the invention. Although the description of the invention is in the context of treatment of blood vessels, and venous and cardiac valve replacement, the invention may also be used in any other body passageways where it is deemed useful. Furthermore, there is no intention to be bound by any expressed or implied theory presented in the preceding technical field, background, brief summary or the following detailed description.

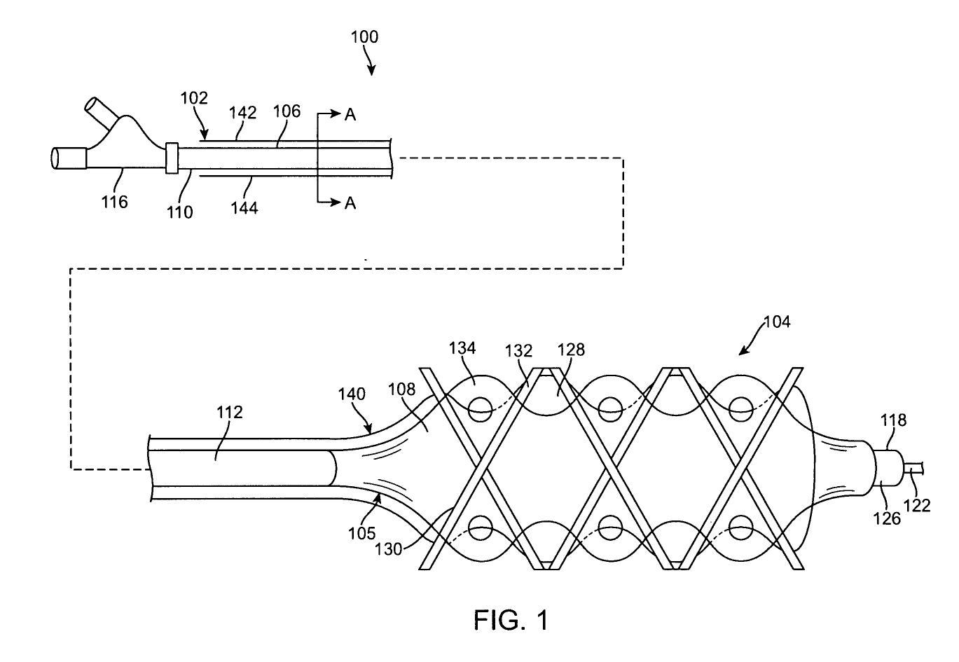

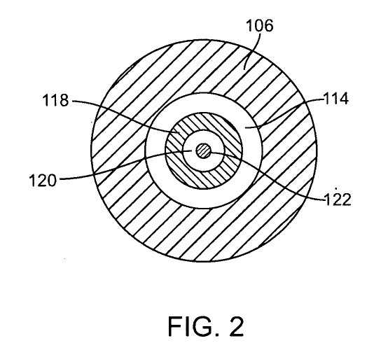

In the embodiment shown in FIGS. 1 and 2, stent delivery system 100 includes an over-the-wire (OTW) catheter configuration having a proximal portion 102 that extends out of the patient and has a hub 116. Distal portion 104 of stent delivery system 100 is positionable at a target location within the vasculature and includes an inflatable balloon 108. Stent delivery system 100 includes an inner guidewire shaft 118 that defines a guidewire lumen 120 extending substantially the entire length of the stent delivery system for accommodating a guidewire 122. Guidewire shaft 118 has a proximal end (not shown) coupled to a proximal guidewire port of hub 116 and a distal end 126 terminating distally of balloon 108 and defining a distal guidewire port. In an embodiment, guidewire shaft 118 may be a flexible tube of a polymeric material, such as, e.g., polyethylene tubing.

In addition, stent delivery system includes an outer shaft component or outer tube 106 having a proximal end 110 coupled to hub 116 and a distal end 112 coupled to balloon 108. In the coaxial catheter construction of the illustrated embodiment, guidewire shaft 118 extends within outer tube 106 such that an annular inflation lumen 114 is defined between an inner surface of outer tube 106 and an outer surface of guidewire shaft 118. Other types of catheter construction are also amendable to the invention, such as, without limitation thereto, a catheter shaft formed by multi-lumen profile extrusion. Inflation lumen 114 extends between proximal and distal ends 110, 112 of outer catheter shaft 106 to allow inflation fluid received through an inflation port of hub 116 to be delivered to balloon 108. As would be understood by one of ordinary skill in the art of balloon catheter design, hub 116 provides a luer hub or other type of fitting that may be connected to a source of inflation fluid and may be of another construction or configuration without departing from the scope of the present invention.

A stent 130 is mounted over a recapture component 105 that is used to partially collapse and reposition stent 130 after deployment. Recapture component 105 is movable between a collapsed configuration and an expanded configuration. In the embodiment depicted in FIGS. 1 and 2, recapture component 105 is inflatable balloon 108. Balloon 108 includes loops 134 formed within the wall of balloon 108 or on an outside surface thereof for receiving a removable elongate, flexible tether or string 140 that connects balloon 108 and stent 130. If it is desired to adjust the positioning of stent 130 after initial deployment, balloon 108 is deflated to at least partially collapse stent 130 so that stent 130 may be repositioned and redeployed. In one embodiment, only partial deflation of balloon 108 is required in order to reposition stent 130.

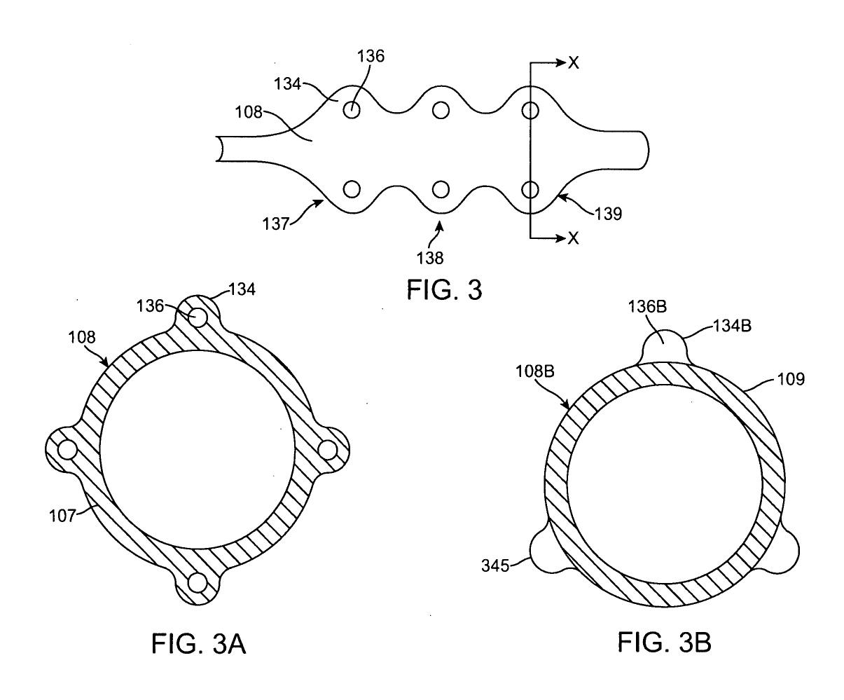

Referring now to FIG. 3, balloon 108 is shown removed from stent delivery system 100. As shown, loops 134 define holes or lumens 136 that are sized to receive tether 140. FIG. 3A is a cross-sectional view of FIG. 3 taken along line X-X, and illustrates that loops 134 may be formed within a wall 107 of balloon 108 during a post-processing step of extruding holes or lumens 136 within the balloon material. In another embodiment depicted in FIG. 3B, which is a cross-sectional view of FIG. 3 according to an alternate embodiment of the invention, loops 134B defining lumens 136B may be formed on an outside surface 109 of balloon 108B during a post-processing step of attaching a wire 345 having a sinusoidal or wavelike shape thereto. For example, the material of the balloon wall may be melted around portions of the wire such that the curves of sinusoidal-shaped wire 345 form loops 134B. Alternatively, sinusoidal-shaped wire 345 may be attached to outside surface 109 of balloon 108B with an adhesive.

Loops 134 are positioned circumferentially around balloon 108. In one embodiment, as shown in FIG. 3B, loops 134B are spaced approximately one hundred twenty degrees apart such that loops 134B occur at three locations around the circumference of balloon 108B. In another embodiment, shown in FIG. 3A, the loops 134 are spaced approximately ninety degrees apart such that they occur at four locations around the circumference of balloon 108. In addition, referring now to FIG. 3, a set of loops 134 is positioned at multiple locations along the length of balloon 108. In one embodiment, a set of loops 134 is located around each of a proximal portion 137, an intermediate portion 138, and a distal portion 139 of balloon 108 such that loops 134 are positioned at three longitudinal locations along the length of balloon 108. However, as will be apparent to one of ordinary skill in the art, the number of sets of loops 134 and their longitudinal and radial spacing may be varied to suit a particular application.

Referring back to FIG. 1, tether 140 includes a first end 142 and a second end 144 that extend out of the patient and may be manipulated by a clinician. As will be explained in more detail herein, tether 140 is woven or laced through loop lumens 136 and openings 128, which are present between stent struts 132, in order to releasably connect balloon 108 and stent 130 together. If it is desired to adjust the positioning of stent 130 after improper deployment, balloon 108, which is still connected to the deployed stent via tether 140, is deflated, which causes at least the partial contraction of stent 130 such that the outer diameter of stent 130 is sufficiently reduced to allow repositioning and redeployment of stent 130. Once stent 130 is properly positioned at the target location and no further adjustments are desired, proximally pulling on one end of tether 140 will disengage tether 140 from balloon 108 and stent 130 for removal. With tether 140 removed, balloon 108 and stent 130 are disconnected and stent delivery system 100 may be removed from the patient.

As previously explained, sets of loops 134 are longitudinally aligned along the length of balloon 108. Tether 140 may be longitudinally woven or laced through loop lumens 136 and openings 128 of each row of longitudinally aligned loops 134. More particularly, tether 140 is laced in a distal direction along the length of the balloon, then back in a proximal direction along the length of the balloon, and so forth until the single line of tether 140 is woven through all rows of longitudinally aligned loops 134. If each set of loops 134 has an odd number of loops (for example, when loops 134B occur at three locations around the circumference of balloon 108B as explained above with reference to FIG. 3B), tether 140 would be “doubled back” after the last woven segment that extends in a distal direction along the length of the balloon in order to proximally return the tether end to extend out of the patient and be manipulated by a clinician.

In another embodiment, multiple tethers 140 may be utilized to simplify releasably connecting balloon 108 and stent 130 together. For example, independent tethers for each row of longitudinally aligned loops 134 may be utilized to simplify the lacing between balloon 108 and stent 130 and to lower friction when removing tethers in situ by pulling the proximal ends thereof. For example, when loops 134B occur at three locations around the circumference of balloon 108B as explained above with reference to FIG. 3B, three individual tethers may be utilized for connecting balloon 108 and stent 130. Each individual tether is woven through loop lumens 136 and openings 128 in a longitudinal manner, as described above with respect to tether 140, and functions in the same manner as tether 140 described herein. More specifically, each individual tether is woven in a distal direction along the length of the balloon and then “doubles back” along the length of the balloon in order to proximally return the tether end to extend out of the patient and be manipulated by a clinician. Similarly, when loops 134 occur at four locations around the circumference of balloon 108 as explained above with reference to FIG. 3A, one, two or four individual tethers may be utilized for connecting balloon 108 and stent 130.

Tether 140 is an elongate flexible filament of biocompatible material having sufficient strength to aid in collapsing stent 130. In one embodiment, tether 140 is a monofilament. In various other embodiments, tether 140 may be a braid of a plurality of filaments of the same or different materials. In still other embodiments, tether 140 may include a braided sheath with a single filament core, or a braided sheath with a braided core. Tether 140 is constructed from a material that will not stretch and/or may be pre-stressed to prevent the tether from elongating during use. Suitable biocompatible materials for tether 140 include but are not limited to nylon, polyethylene, and polyester, as well as other high strength suture materials. In an embodiment, tether 140 may include one or more pre-stretched filaments of an ultra high molecular weight polyethylene, such as a filament made from DYNEEMA fibers. In an embodiment, tether 140 may also include a hydrophilic coating to aid in removing the tether from balloon 108 and stent 130 after proper deployment. Various embodiments hereof include tethers having diameters in the range of 0.015 inches and 0.050 inches in diameter. However, depending on the application, tethers having a diameter smaller than 0.015 inches or larger than 0.050 inches may be used.

Stent 130 may have any suitable configuration known in the art. For example, as shown in FIG. 1, stent 130 may have cylindrically-shaped tubular body formed by a plurality of adjacent connected stent struts 132. One of ordinary skill in the art will appreciate that stent 130 can have any number of stent struts 132 depending upon the desired length of stent 130.

As described in the embodiment of FIG. 1, tether 140 is woven between loop lumens 136 and stent strut openings 128 in order to connect balloon 108 and stent 130. In an alternate embodiment, stent struts 132 may be formed with appropriately sized “pinholes” therein for receiving tether 140 therethrough. In yet another embodiment, a laser-cut stent may be utilized as stent 130 with appropriately sized “pinholes” stamped or otherwise formed therein for receiving tether 140.

In any of the embodiments described herein, the stent may include a valve located therein capable of blocking flow in one direction. The valve may be sealingly and permanently attached to the interior surface of the stent and/or graft material enclosing or lining the stent. The graft material may be a low-porosity woven fabric, such as polyester, Dacron fabric, or PTFE, which creates a one-way fluid passage when attached to the stent. The valve may be a bovine or porcine valve treated and prepared for use in a human, or may be a mechanical valve or a synthetic leaflet valve. For example, the stent may be a percutaneously implanted bovine or porcine valve treated and prepared for use in a human and sewn inside a laser-welded stent such as that described in U.S. Pat. No. 5,957,949, the contents of which were previously incorporated by reference. When a tissue valve is located within the stent, care should be taken that tether 140 does not pierce the tissue.

In one embodiment, stent 130 is balloon expandable. Deployment of balloon expandable stent 130 is accomplished by tracking stent delivery system 100 through the vascular system of the patient until stent 130 is located within a stenosis at a predetermined treatment site. Once positioned, a source of inflation fluid is connected to an inflation port of hub 116 such that balloon 108 is inflated to expand stent 130 by the radial force of the balloon as is known to one of ordinary skill in the art. When fully expanded, stent 130 contacts the vascular wall to maintain the opening thereof. Stent deployment can be performed following treatments such as angioplasty, or during initial balloon dilation of the treatment site, which is referred to as primary stenting.

In another embodiment, stent 130 may be self-expanding such that balloon 108 is used only for repositioning of stent 130 as described above. Deployment of stent 130 may be facilitated by utilizing shape memory characteristics of a material such as nickel-titanium (nitinol). More particularly, shape memory metals are a group of metallic compositions that have the ability to return to a defined shape or size when subjected to certain thermal or stress conditions. Shape memory metals are generally capable of being deformed at a relatively low temperature and, upon exposure to a relatively higher temperature, return to the defined shape or size they held prior to the deformation. This enables the stent to be inserted into the body in a deformed, smaller state so that it assumes its “remembered” larger shape once it is exposed to a higher temperature, i.e., body temperature or heated fluid, in vivo. Thus, self-expanding stent 130 can have two states of size or shape, a contracted or compressed configuration sufficient for delivery to the treatment site and a deployed or expanded configuration having a generally cylindrical shape for contacting the vessel wall. In another embodiment in which stent 130 is self-expanding, stent 130 may be constructed out of a spring-type or superelastic material. When a self-expanding stent is used with the stent delivery system 100, a sheath (not shown) may be provided to surround and contain self-expanding stent 130 in a contracted or compressed position. Once self-expanding stent 130 is in position at a site of a stenotic lesion, the sheath may be retracted, thus releasing stent 130 to assume its expanded or deployed configuration.

Some examples of self-expanding and balloon-expandable stents that are suitable for use in embodiments of the present invention are shown in U.S. Pat. No. 4,733,665 to Palmaz, U.S. Pat. No. 4,800,882 to Gianturco, U.S. Pat. No. 4,886,062 to Wiktor, U.S. Pat. No. 5,133,732 to Wiktor, U.S. Pat. No. 5,292,331 to Boneau, U.S. Pat. No. 5,421,955 to Lau, U.S. Pat. No. 5,776,161 to Globerman, U.S. Pat. No. 5,935,162 to Dang, U.S. Pat. No. 6,090,127 to Globerman, U.S. Pat. No. 6,113,627 to Jang, U.S. Pat. No. 6,663,661 to Boneau, and U.S. Pat. No. 6,730,116 to Wolinsky et al., each of which is incorporated by reference herein in its entirety.

In any of the embodiments described herein, stent delivery system 100 may be modified to be of a rapid exchange (RX) catheter configuration without departing from the scope of the present invention such that guidewire shaft 118 extends within only distal portion 104. In such an embodiment, a proximal portion of outer catheter shaft 106 may include a metal hypotube with a guidewire transition area having a proximal guidewire port being positioned proximal of balloon 108.

Repositioning of stent 130 may be facilitated by the use of a guide catheter having reinforcement, such as a metal band, at the distal tip thereof. The guide catheter is placed over proximal shaft outer tube 106 and tether 140 with the distal reinforcement positioned slightly proximal of balloon 108. The guide catheter prevents deformation of outer tube 106 when tether 140 is pulled proximally to collapsible stent 130, with the distal reinforcement protecting the guide catheter's distal tip from being cut by tether 140 during the collapsing procedure. In an embodiment, the guide catheter includes a braided layer for shaft support, and includes a reinforced distal tip, such as a metal band surrounding the distal tip as described above, to avoid damage to the guide catheter tip when tension is applied to tether 140. Suitable embodiments of the guide catheter may include the EXPORT guide catheter or the LAUNCHER guide catheter, both manufactured by Medtronic, Inc. of Minneapolis, Minn.

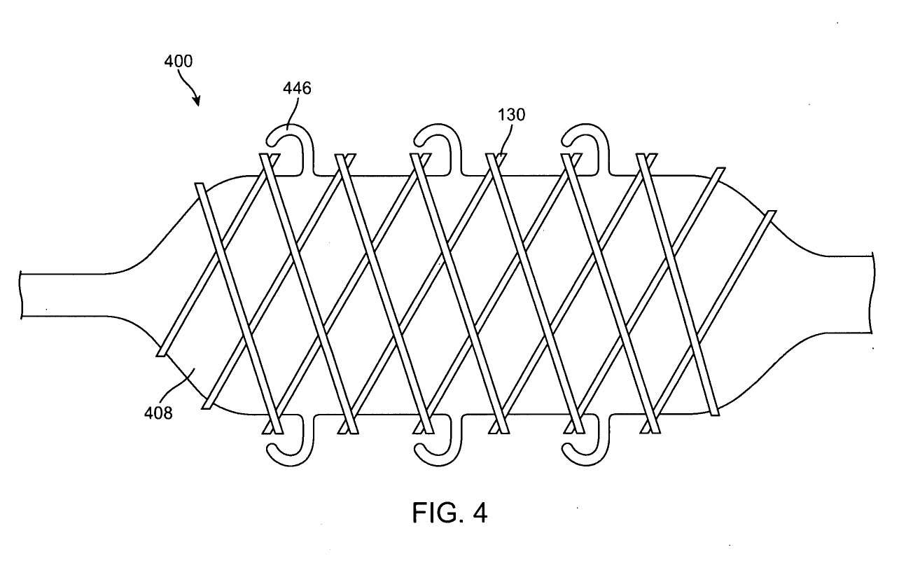

A distal portion of another embodiment of stent delivery system 400 is illustrated in FIG. 4. Similar to the previous embodiment of FIGS. 2 and 3, an inflatable balloon 408 of stent delivery system 400 is used as the recapture component for at least partially collapsing and repositioning stent 130 after improper deployment. However, in the embodiment of FIG. 4, no tether is required for connecting balloon 408 and stent 130. Rather, balloon 408 includes hooks 446 integrally formed from the wall of balloon 408 to extend from an outside surface thereof for directly engaging stent 130. Hooks 446 may be formed to curve around the stent struts of stent 130, or may curve within holes formed within the stent body of stent 130 as described above.

In an embodiment, hooks 446 may be wires having a hook or curved shape that are sufficiently stiff to engage stent 130. Wire hooks 446 may be attached to balloon 408 during a post-processing step, such as by melting the material of the balloon wall around each wire such that the hook of the wire radially extends from an outside surface of the balloon, or alternatively by adhesive. Hooks 446 may be positioned circumferentially around balloon 408 with sets of hooks 446 longitudinally spaced along the length of balloon 408 as described above with respect to loops 134. As shown in FIG. 4, hooks 446 point in a proximal direction. Such proximal orientation of hooks 446 may aid in easing the initial deployment of a self-expanding stent 130 from a delivery catheter, and may be less traumatic to the vasculature if balloon 408 is required to be advanced after initial deployment of self-expanding stent 130. In another embodiment (not shown), hooks 446 may alternatively point in a distal direction.

If it is desired to adjust the positioning of stent 130 after initial deployment, balloon 408 is deflated with hooks 446 engaged into stent 130. As stent 130 is directly connected to balloon 408, deflation of balloon 408 urges stent 130 into at least a partially collapsed configured such that stent 130 may be repositioned and redeployed. Once stent 130 is positioned precisely at the target location and no further adjustments are desired, balloon 408 and stent 130 are disconnected such that stent delivery system 400 may be removed from the patient. In order to remove stent delivery system 400, balloon 408 may be manufactured to deflate in a particular way such that hooks 446 disengage stent 130.

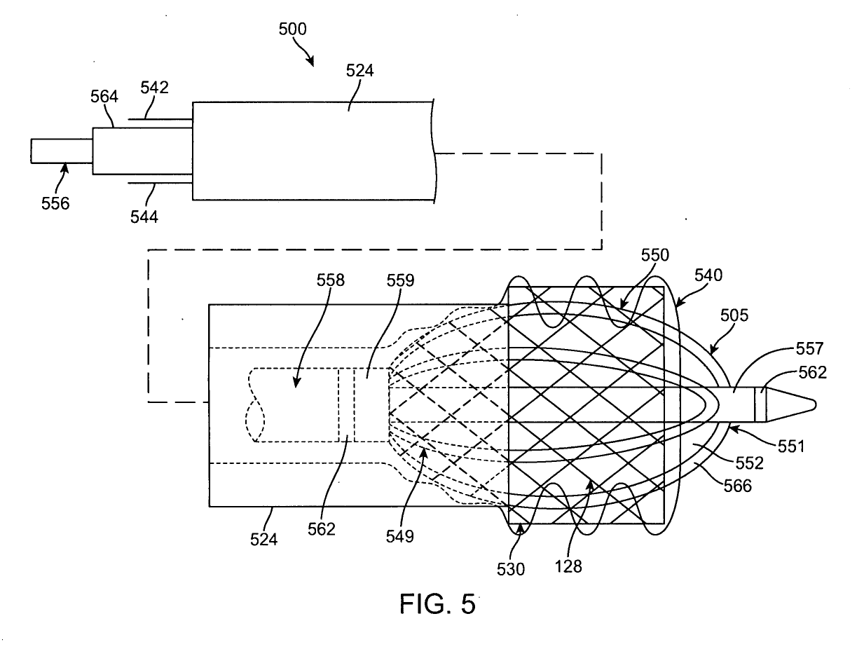

Another embodiment of stent delivery system 500 is illustrated in FIG. 5. Unlike the previous embodiments, a mechanically-expandable tubular component 550 rather than a balloon is used as recapture component 505 to collapse and reposition stent 530 after improper deployment. Stent 530 is self-expanding such that stent delivery system 500 does not include a balloon. In order to deploy self-expanding stent 530, a retractable sheath 524 constrains stent 530 during delivery and is proximally retracted in order to allow stent 530 to radially expand. In FIG. 5, sheath 524 is shown partially withdrawn with stent 530 partially expanded. In another embodiment (not shown), stent 530 is balloon-expandable and tubular component 550 is positioned between stent 530 and the balloon, which is utilized only for expanding the stent and not as the recapture component. Expandable tubular component 550 includes openings 552 therein that receive a tether or string 540 for connecting tubular component 550 and stent 530. If it is desired to adjust the positioning of stent 530 after improper deployment, tubular component 550 is operated to collapse or contract stent 530 for repositioning and redeployment.

More particularly, tubular component 550 is expanded and contracted by relative movement between an inner tube 556 and an outer tube 558 that are operably attached to tubular component 550, as discussed below. Tubular component 550, inner tube 556, and outer tube 558 cooperate to provide an expansion framework that is controlled to be movable between a reduced-diameter or collapsed configuration and an enlarged-diameter expanded configuration. Inner tube 556 extends within the lumen of outer tube 558, and is movable in an axial direction along and relative to outer tube 558. Coaxial tubes 556, 558 extend the length of stent delivery system 500 such that the proximal ends thereof (not shown) extend out of the patient and may be manipulated by a clinician. A proximal end 549 of tubular component 550 is attached to a distal end 559 of outer tube 558 and a distal end 551 of tubular component 550 is attached to a distal end 557 of inner tube 556. While holding a proximal end 564 of outer tube 558 fixed, inner tube 556 may be proximally retracted within outer tube 558. When inner tube 556 is proximally retracted, the attachment point between tubular component 550 and outer tube 558 remains fixed such that tubular component 550 radially expands.

Although embodiments are described with inner tube 556 being movable relative to outer tube 558 to expand tubular component 550, it should be apparent to one of ordinary skill in the art that tubular component 550 is expanded by shortening the distance between ends 549, 551 thereof. Thus, in another embodiment, tubular component 550 may be expanded by distally advancing outer tube 558 while holding inner tube 556 stationary. In addition, tubular component 550 may be expanded by a combination of distally advancing outer tube 558 and proximally retracting inner tube 556. Tubes 556, 558 may include radiopaque markers, such as metal annular bands 562, at distal ends 557, 559, respectively, to aid in fluoroscopic visualization of system 500 during delivery and stent deployment.

Tubular component 550 may be attached to outer tube 558 and inner tube 556 in any suitable manner known in the art. For example, the connection may be formed by welding, such as by resistance welding, friction welding, laser welding or another form of welding such that no additional materials are used to connect tubular component 550 to tubes 556, 558. Alternatively, tubular component 550 can be connected to tubes 556, 558 by soldering, by the use of an adhesive, by the addition of a connecting element there between, or by another mechanical method.

Referring back to FIG. 5, as previously mentioned, tether or string 540 connects tubular component 550 and stent 530 such that when tubular component 550 is transformed into its collapsed configuration within the deployed stent, an outer diameter of stent 130 will be reduced and an outer surface of stent 130 will be moved out of apposition with the vessel wall to allow repositioning thereof. Tether 540 is woven between tubular component openings 552 and stent strut openings 528 in order to connect tubular component 550 and stent 530. Similar to tether 140 described above, tether 540 includes a first end 542 and a second end 544 that extend out of the patient and may be manipulated by a clinician. If it is desired to adjust the positioning of stent 530 after improper deployment, tubular component 550 is collapsed which in turn at least partially contracts stent 530, which may then be repositioned and redeployed. Once stent 530 is positioned precisely at the target location and no further adjustments are desired, pulling or retracting one end of tether 540 will disengage tether 540 from tubular component 550 and stent 530. With tether 540 removed, tubular component 550 and stent 530 are disconnected and stent delivery system 500 may be removed from the patient.

Tubular component 550 may have any suitable configuration known in the art. For example, as shown in FIG. 5, tubular component 550 may include a plurality of strands or flat ribbons 566 that are approximately equal in length, extend generally parallel to the blood flow, and are uniformly and circumferentially arranged about a longitudinal axis of system 500. One of ordinary skill in the art will appreciate that tubular component 550 may include any number of ribbons. For example, tubular component 550 may include between two and eight ribbons that longitudinally extend when expanded. In yet another embodiment, the flat ribbons or strands may extend in a helical manner around the longitudinal axis of system 500.

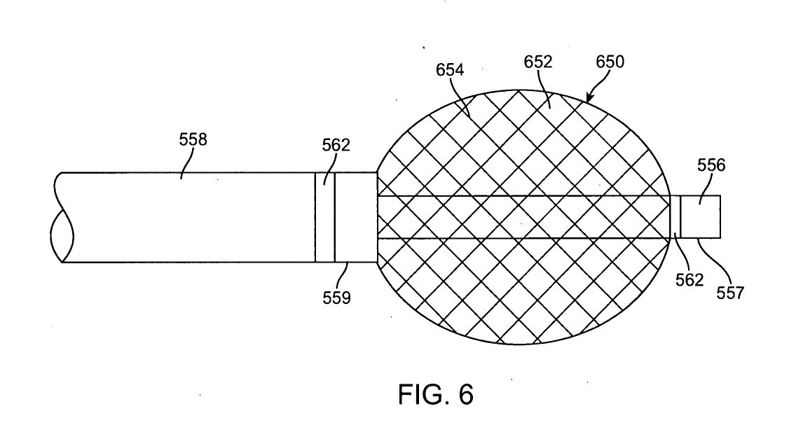

FIG. 6 illustrates a tubular component 650 according to another embodiment hereof that is removed from the stent delivery system for clarity. Tubular component 650 is formed from a stamped or braided mesh 654. Mesh 654 has openings 652 therein that are of a sufficient size to receive a removable tether, as described in the previous embodiments.

Mechanically-expandable tubular components according to embodiment hereof are preferably constructed of implantable polymeric or metallic materials having good mechanical strength while maintaining a minimized delivery profile. Non-exhaustive examples of polymeric materials for the tubular component are polyurethane, polyethylene terephalate (PET), nylon, polyethylene, PEBAX, or combinations of any of these, either blended or co-extruded. Non-exhaustive examples of metallic materials for the tubular component are stainless steel, cobalt based alloys (605L, MP35N), titanium, tantalum, superelastic nickel-titanium alloy, or combinations of any of these.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A stent delivery system for repositioning an improperly deployed stent in situ, the system comprising:
a recapture component movable between a collapsed configuration and an expanded configuration;
an expandable stent mounted over the recapture component, wherein the stent remains connected to the recapture component after deployment such that when the recapture component is moved to the collapsed configuration the stent is at least partially contracted.

2. The stent delivery system of claim 1, wherein at least one removable tether connects the recapture component to the stent.

3. The stent delivery system of claim 2, wherein the at least one tether is medical grade fishing line.

4. The stent delivery system of claim 1, wherein the recapture component is an inflatable balloon.

5. The stent delivery system of claim 4, wherein the inflatable balloon includes a plurality of loops, each loop having a lumen sized to receive at least one removable tether.

6. The stent delivery system of claim 5, wherein the at least one tether is woven between the loop lumens and openings between stent struts of the stent to connect the balloon and stent.

7. The stent delivery system of claim 5, wherein the loops are integrally formed from material of the balloon.

8. The stent delivery system of claim 5, wherein the loops are portions of a sinusoidal or wavelike wire attached to the balloon.

9. The stent delivery system of claim 5, wherein the loops are circumferentially spaced from each other and are positioned at least three locations around the circumference of the balloon.

10. The stent delivery system of claim 5, wherein the loops are longitudinally spaced from each other and positioned at least a proximal portion, an intermediate portion, and a distal portion along the length of the balloon.

11. The stent delivery system of claim 1, wherein the stent is balloon expandable.

12. The stent delivery system of claim 1, wherein the stent is self-expanding.

13. The stent delivery system of claim 4, wherein the inflatable balloon includes a plurality of hooks formed thereon for directly engaging the stent, thereby connecting the balloon to the stent.

14. The stent delivery system of claim 2, wherein the recapture component is an expandable tubular component having a plurality of openings formed therein for receiving the at least one removable tether that connects the tubular component to the stent.

15. The stent delivery system of claim 14, further comprising:
an outer tube having a distal end coupled to a proximal end of the tubular component;
an inner tube slidably positioned within the outer tube, wherein a distal end of the tubular component is attached to a distal end of the inner tube; and
the tubular component is movable between a collapsed configuration and an expanded configuration by relative movement between the inner tube and the outer tube.

16. The stent delivery system of claim 15, wherein the expandable tubular component is selected from the group consisting of a braided or stamped mesh and a plurality of parallel ribbons.

17. A stent delivery system for repositioning a deployed stent in situ comprising:
an inflatable balloon, wherein the inflatable balloon includes a plurality of loops defining a plurality of lumens on an outside surface of the balloon;
a deployable stent mounted over the inflatable balloon; and
at least one removable tether woven between the loop lumens and the stent to connect the balloon and the stent in such a manner that the stent, after deployment, at least partially collapses when the balloon is deflated.

18. A method of repositioning a deployed stent in situ, the method comprising the steps of:
deploying a stent within a body lumen with a stent delivery system, the system having a recapture component connected to the stent and movable between a collapsed configuration and an expanded configuration;
at least partially collapsing the recapture component to the collapsed configuration, thereby at least partially collapsing the stent;
repositioning the stent within the body lumen;
redeploying the stent within the body lumen with the stent delivery system;
disconnecting the redeployed stent from the recapture component; and
removing the stent delivery system from the body lumen.

19. The method of claim 18, wherein the recapturing component is an inflatable balloon.

20. The method of claim 18, wherein the recapture component is an expandable tubular component having a plurality of openings formed therein for receiving a removable tether that connects the tubular component to the stent.
